(19) United States
(12) Patent Application Publication
(10) Pub. No.: US 2023/0372638 A1
(43) Pub. Date: Nov. 23, 2023

(54) ATOMIZER AND ATOMIZATION METHOD FOR ADAPTIVELY GENERATING PHARMACEUTICAL AEROSOLS OF DIFFERENT PARTICLE SIZES

(71) Applicant: NATIONAL TSING HUA UNIVERSITY, Hsinchu City (TW)

(72) Inventors: CHI-SHUO CHEN, HSINCHU CITY (TW); HUI-LING LIN, HSINCHU CITY (TW); SHENG-TING HUNG, HSINCHU CITY (TW); GENG-YUE LI, HSINCHU CITY (TW); MEGHA JHUNJHUNWALA, HSINCHU CITY (TW)

(21) Appl. No.: 17/850,635

(22) Filed: Jun. 27, 2022

(30) Foreign Application Priority Data
May 20, 2022 (TW) 111118808

(51) Int. Cl. A61M 11/00; A61M 16/00; G06N 3/02; G06K 9/00

(52) U.S. Cl. CPC A61M 11/005; A61M 16/024; G06N 3/02; G06K 9/0051

(57) ABSTRACT

An atomizer for adaptively generating pharmaceutical aerosols of different particle sizes is illustrated, which comprises a sensor module, a waveform pattern determination module, a high-frequency oscillation circuit, a signal modulation module, a piezoelectric device and a porous screen. The sensor module senses a body state of the patient, wherein the body state includes a breathing state. The waveform pattern determination module generates a modulation control signal according to the body state. The high-frequency oscillation circuit provides a high-frequency oscillation signal. The signal modulation module modulates the high-frequency oscillation signal by using the modulation control signal to generate a vibration signal. The piezoelectric device vibrates according to the vibration signal. According to vibration of the piezoelectric device, the porous screen presses the liquid medicine through a plurality of holes of the porous screen to generate a plurality of pharmaceutical aerosols.

### BACKGROUND

### Technical Field

The present disclosure is related to an atomizer and an atomization method for generating a plurality of pharmaceutical aerosols, and in particularly, to an atomizer and an atomization method for adaptively generating a plurality of pharmaceutical aerosols with different particle sizes according to a body state of the patient, treatment medicine for the patient and other factors.

### Related Art

Pharmaceutical aerosols is an atomized liquid with medicine drug particles, and the pharmaceutical aerosols can enter the upper respiratory tract, lower respiratory tract and lungs of the human body, and be absorbed by the upper respiratory tract, lower respiratory tract and lungs to further treat specific diseases by using the medicine drug particles. The particle sizes of pharmaceutical aerosols that can be absorbed by different parts of the upper respiratory tract, lower respiratory tract and lungs are not the same. Usually, the particle size of pharmaceutical aerosols is expressed by the mass medium aerodynamic diameter (MMAD).

For example, the MMAD of the pharmaceutical aerosols which can be absorbed by the upper respiratory tract (such as, nose, throat, trachea) is about 5 μm to 50 μm, even larger than 50 μm; the MMAD of the pharmaceutical aerosols which can be absorbed by the lower respiratory tract is about 2 μm to 5 μm; the MMAD of the pharmaceutical aerosols which can be absorbed by the alveolar area is about 1 μm to 3 μm; and the MMAD of the pharmaceutical aerosols which can be absorbed by the parenchyma of lungs is less than 0.1 μm.

For patients with different symptoms, it is necessary to send drug particles to different tissues of the patient. However, at present, the atomizer can only produce pharmaceutical aerosols of a specific particle size, so it is necessary to use a plurality of atomizers that produce pharmaceutical aerosols of different particle sizes to meet various therapeutic needs. On the other hand, there are still differences in physique between patients. Therefore, pharmaceutical aerosols for the same tissue may still have differences in particle size to meet the purpose of precision treatment, or, because of different medicine drugs, the particle size of the pharmaceutical aerosols must vary to make the pharmaceutical aerosols be absorbed by the patient.

For example, for the treatment of throat inflammation, the pharmaceutical aerosols of the 40 μm particle size can be effectively absorbed by a throat of a first patient, however, the pharmaceutical aerosols of the 40 μm particle size cannot be effectively absorbed by a throat of a second patient, but the pharmaceutical aerosols of the 45 μm particle size can be effectively absorbed by the throat of the second patient. For example, for the treatment of infections in the alveolar region, the pharmaceutical aerosols of the 2 μm particle size which contains a first medicine drug can be effectively absorbed by the alveolar region of the first patient, the pharmaceutical aerosols of the 2 μm particle size which contains a second medicine drug cannot be effectively absorbed by the alveolar region of the first patient, but the pharmaceutical aerosols of the 3 μm particle size which contains the second medicine drug cannot be effectively absorbed by the alveolar region of the first patient. In short, there are needs of an atomizer and an atomization method for adaptively generating a plurality of pharmaceutical aerosols with different particle sizes according to a body state of the patient, treatment medicine for the patient and other factors.

### SUMMARY

One main objective of the present disclosure is to provide an atomizer and an atomization method for adaptively generating a plurality of pharmaceutical aerosols with different particle sizes according to a body state of the patient, treatment medicine for the patient and other factor. According to the body state of different patients, different diseases, different medicine drugs, different humidity degrees or temperatures and other factors, the atomizer and atomization method of the embodiment of the present disclosure will dynamically generate pharmaceutical aerosols with different particle sizes, thereby achieving the goal of precision treatment.

The present disclosure provides an atomizer for adaptively generating a plurality of pharmaceutical aerosols with deferment particle sizes, which comprises: a sensor module, configured to sense a body state of a patient, wherein the body state comprises a breathing state; a waveform pattern determination module, electrically connected to the sensor module, configured to generate a modulation control signal according to the body state; a high-frequency oscillation circuit, configured to provide a high-frequency oscillation signal; a signal modulation module, electrically connected to the waveform pattern determination module and the high-frequency oscillation circuit, configured to module the high-frequency oscillation signal by using the modulation control signal to generate a vibration signal; a piezoelectric device, electrically connected to the signal modulation module, configured to vibrate according to the vibration signal; and a porous screen, physically connected to the piezoelectric device, configured to presses liquid medicine through a plurality of holes of the porous screen to generate the plurality of pharmaceutical aerosols according to vibration of the piezoelectric device, wherein the pharmaceutical aerosols has the particle size(s) corresponding to the modulation control signal.

According to the features of the above atomizer, the atomizer further comprises: a data transmission interface, electrically connected to the waveform pattern determination module, configured to obtain auxiliary information; wherein the signal modulation module generates the modulation control signal according to the body state and the auxiliary information, the auxiliary information comprises at least one of a medicine drug type and a disease type, and the body state further comprises at least of a facial expression, a heartbeat rate, a heart rhythm, a blood pressure, a glucose and body temperature of the patient.

According to the features of the above atomizer, the atomizer further comprises: a manual switch, electrically connected to the waveform pattern determination module, configured to generate a switch control signal according to manipulation of the patient or a manipulator, wherein the waveform pattern determination module generates the modulation control signal according to the switch control signal.

According to the features of the above atomizer, the atomizer further comprises: a direct current/alternative current (DC/AC) converter, electrically connected to the high-frequency oscillation circuit, configured to convert an AC input voltage to a DC input voltage, so as to provide the DC input voltage to the high-frequency oscillation circuit, wherein a frequency of the high-frequency oscillation signal generated by the high-frequency oscillation circuit is corresponding to the DC input voltage; and a power amplifier, electrically connected between the piezoelectric device and the signal modulation module, configured to amplify the vibration signal and output the amplified vibration signal to the piezoelectric device.

According to the features of the above atomizer, the waveform pattern determination module comprises: a signal processing module, electrically connected to the sensor module, configured to perform noise filtering process on a sensing signal of the body state; a core computing module, electrically connected to the signal processing module, configured to calculate a waveform pattern according to the sensing signal on which the noise filtering process is performed; and a modulation control signal generation module, electrically connected to the core computing module and the signal modulation module, configured to generate the modulation control signal according to the calculated waveform pattern.

According to the features of the above atomizer, the core computing module comprises a neural network based classifier, and the neural network based classifier is configured to calculate a waveform pattern according to the sensing signal on which the noise filtering process is performed.

According to the features of the above atomizer, the sensor module comprises a microphone module.

According to the features of the above atomizer, the atomizer further comprises: a container, configured to accommodate the liquid medicine; and a nozzle, disposed on an outer surface of the container and arranged corresponding to the holes, configured to spray the plurality of the pharmaceutical aerosols.

According to the features of the above atomizer, the signal modulation module comprises: a mixer, electrically connected to the high-frequency oscillation circuit and the waveform pattern determination module, configured to mix the modulation control signal and the high-frequency oscillation signal to generate a mixed signal; and a filter module filter module, electrically connected to the mixer and the piezoelectric device, configured to perform an intermediate frequency filtering process on the mixed signal to generate the vibration signal.

According to the features of the above atomizer, the modulation control signal is one or combination of a square wave, a triangle wave and a sinuous wave.

The present disclosure provides an atomization method for adaptively generating a plurality of pharmaceutical aerosols with deferment particle sizes, which comprises:

using a sensor module to sense a body state of a patient, wherein the body state comprises a breathing state; using a waveform pattern determination module to generate a modulation control signal according to the body state; using a high-frequency oscillation circuit to provide a high-frequency oscillation signal; using a signal modulation module to module the high-frequency oscillation signal by using the modulation control signal to generate a vibration signal; and using a piezoelectric device to vibrate according to the vibration signal so as to make a porous screen presses liquid medicine through a plurality of holes of the porous screen to generate the plurality of pharmaceutical aerosols according to vibration of the piezoelectric device, wherein the pharmaceutical aerosols has the particle size(s) corresponding to the modulation control signal.

According to the features of the above atomization method, the atomization method further comprises: using a data transmission interface to obtain auxiliary information; wherein the signal modulation module generates the modulation control signal according to the body state and the auxiliary information, the auxiliary information comprises at least one of a medicine drug type and a disease type, and the body state further comprises at least of a facial expression, a heartbeat rate, a heart rhythm, a blood pressure, a glucose and body temperature of the patient.

According to the features of the above atomization method, the atomization method further comprises: using a manual switch to generate a switch control signal according to manipulation of the patient or a manipulator, wherein the waveform pattern determination module generates the modulation control signal according to the switch control signal.

According to the features of the above atomization method, the atomization method further comprises: using a direct current/alternative current (DC/AC) converter to convert an AC input voltage to a DC input voltage, so as to provide the DC input voltage to the high-frequency oscillation circuit, wherein a frequency of the high-frequency oscillation signal generated by the high-frequency oscillation circuit is corresponding to the DC input voltage; and using a power amplifier to amplify the vibration signal and output the amplified vibration signal to the piezoelectric device.

According to the features of the above atomization method, the step of using the waveform pattern determination module to generate the modulation control signal according to the body state comprises: using a signal processing module to perform noise filtering process on a sensing signal of the body state; using a core computing module to calculate a waveform pattern according to the sensing signal on which the noise filtering process is performed; and using a modulation control signal generation module to generate the modulation control signal according to the calculated waveform pattern.

According to the features of the above atomization method, the core computing module comprises a neural network based classifier, and the neural network based classifier is configured to calculate a waveform pattern according to the sensing signal on which the noise filtering process is performed.

According to the features of the above atomization method, the sensor module comprises a microphone module.

According to the features of the above atomization method, the atomization method further comprises: using a container to accommodate the liquid medicine; and using a nozzle to spray the plurality of the pharmaceutical aerosols.

According to the features of the above atomization method, the step of using the signal modulation module to module the high-frequency oscillation signal by using the modulation control signal to generate the vibration signal comprises: using a mixer to mix the modulation control signal and the high-frequency oscillation signal to generate a mixed signal; and using a filter module filter module to perform an intermediate frequency filtering process on the mixed signal to generate the vibration signal.

According to the features of the above atomization method, the modulation control signal is one or combination of a square wave, a triangle wave and a sinuous wave.

To sum up, the present disclosure provides an atomizer and an atomization method, and both of them can obtain different factors, and dynamically adjust the waveform pattern of the high-frequency oscillation signal, such that the particle size(s) of the pharmaceutical aerosols is adaptively adjusted, and the pharmaceutical aerosols with the specific medicine drug particles can be effectively absorbed by the specific tissue by the patient, and the goal of precision treatment can be achieved.

### BRIEF DESCRIPTIONS OF DRAWINGS

FIG. 1 is a schematic diagram showing use of an atomizer according to an embodiment of the present disclosure.

FIG. 2 is a schematic diagram showing function blocks of an atomizer according to an embodiment of the present disclosure.

FIG. 3 is a schematic diagram showing function blocks of a waveform pattern determination module according to an embodiment of the present disclosure.

FIG. 4 is a schematic diagram showing generation of pharmaceutical aerosols by using a piezoelectric device and a porous screen according to an embodiment of the present disclosure.

FIG. 5 is a flow chart of an atomization method according to an embodiment of the present disclosure.

### DETAILS OF EXEMPLARY EMBODIMENTS

For the person with the ordinary skill in the art to understand the technical features, the content, and the advantages of the present disclosure, as well as the benefits that the present disclosure can achieve, the present disclosure is hereinafter described in details with the accompanying drawings and the expression in the form of embodiments. The drawings used in the description are only for illustration and as assistance to the specification, which may not be necessarily true in scale and precise configuration after the implementation of the present disclosure. Therefore, one should not interpret according to the scale and configuration in the accompanying drawings to limit the claims of the present disclosure on practical implementation.

When using pharmaceutical aerosols to treat the disease of the patient, there are several factors to be considered, so as to generate pharmaceutical aerosols with different particle sizes for treatment of the patient. That is, to achieve the precision treatment, it needs to adaptively to generate the pharmaceutical aerosols with different particle sizes, i.e. the particle size should be adaptively determined. Accordingly, the present disclosure provides an atomizer and an atomization method, and both of them acquire several factors (such as, a breathing state of the patient sensed by the microphone module and a medicine drug type for the patient transmitted via the wireless communication interface). Then, both of them adjust the waveform pattern of the high-frequency oscillation signal accordingly. For example, a high-frequency oscillation signal is generated to act as a carrier, and a modulation control signal is generated to modulate a frequency, amplitude or phase of the high-frequency oscillation signal, such that a vibration signal is accordingly generated to the piezoelectric device for vibrating. Thus, the pharmaceutical aerosols of the medicine drug particles can be effectively absorbed by the specific tissue of the patient.

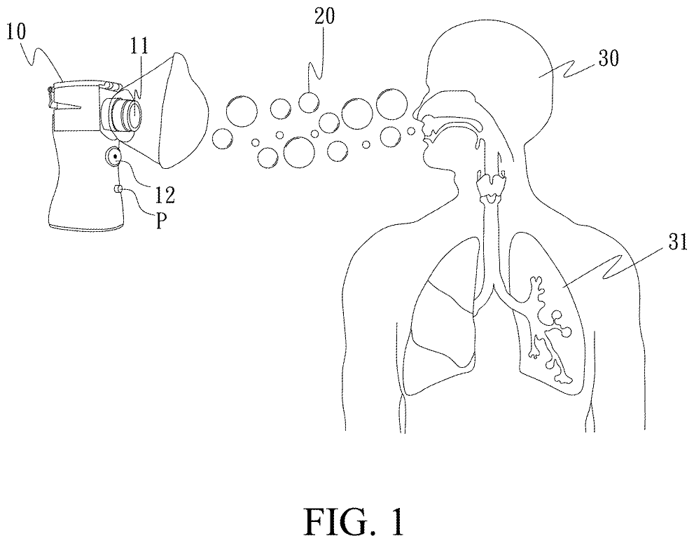

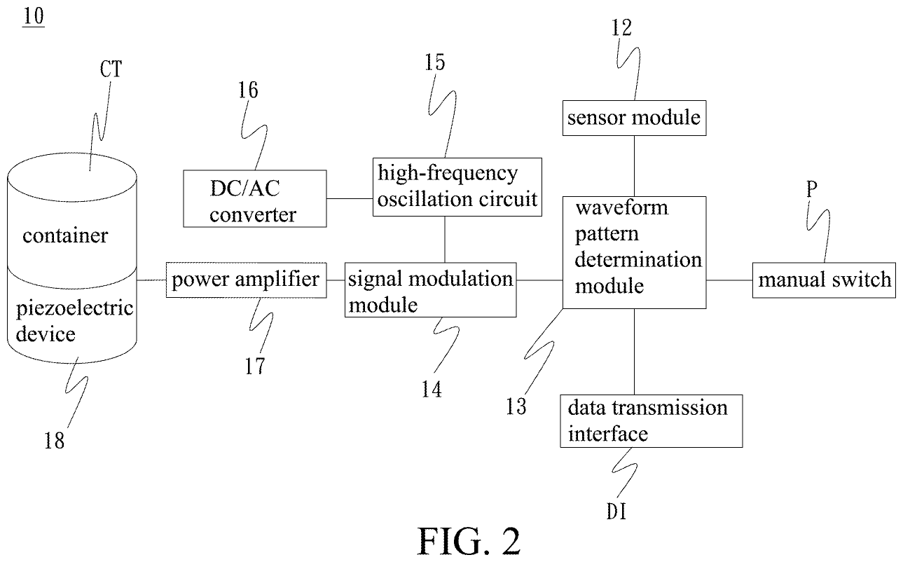

Refer to FIG. 1, and FIG. 1 is a schematic diagram showing use of an atomizer according to an embodiment of the present disclosure. The atomizer 10 for adaptively generating a plurality of pharmaceutical aerosols 20 with different particle sizes is configured to take factor in consideration, so as generate the pharmaceutical aerosols 20 with one particle size or multiple particle sizes to treat the disease of the patient 30. That is, the particle size(s) of the pharmaceutical aerosols 20 is determined by at least one of the several factors. Generally, the factors to be considered can be sensed by using the sensor module 12 of the atomizer 10 or obtained by other manners. For example, the atomizer 10 is wirelessly or wiredly connected to a source of auxiliary information, so as to obtain the auxiliary information as the factors to be considered. In addition, the nozzle 11 of the atomizer 10 is to spray the pharmaceutical aerosols 20 generated by the atomizer 10 to the upper respiratory tract of the patient 30, such that the patient 30 can inhale the pharmaceutical aerosols 20, wherein the nozzle 11 can be disposed on the outer surface of the container CT of the atomizer 10, and the container CT is shown in FIG. 2. Then, the pharmaceutical aerosols 20 can be effectively absorbed by the upper respiratory tract, the lower respiratory tract, the alveolar area or the parenchyma. For example, the medicine drug particles in the pharmaceutical aerosols 20 are effectively absorbed by the tissue of the lungs of the patient 30, and thus the atomizer 10 can perform specific precision treatment on the patient 30.

Specifically, the atomizer 10 is configured to dynamically generate the plurality of pharmaceutical aerosols 20 with different particle sizes at different times according at least one of the factors; or alternatively, the atomizer 10 is configured to generate the plurality of pharmaceutical aerosols 20 with different particle sizes at the same time; or alternatively, the atomizer 10 is configured to generate the plurality of pharmaceutical aerosols 20 with different particle sizes at a first time and the plurality of pharmaceutical aerosols 20 with other different particle sizes at a second time, i.e., the distribution of the particle sizes of the pharmaceutical aerosols 20 of the first time is not the same as that of the second time. Regarding the last one of the above designs, the pharmaceutical aerosols 20 of the first time comprises 50% pharmaceutical aerosols 20 with the first particle size and 50% pharmaceutical aerosols 20 with the second particle size, and the pharmaceutical aerosols 20 of the first time comprises 25% pharmaceutical aerosols 20 with the first particle size, 25% pharmaceutical aerosols 20 with the second particle size and 50% pharmaceutical aerosols 20 with the third particle size. The particle size of the pharmaceutical aerosols 20 can be the MMAD, and the present disclosure is not limited, and in another embodiment, the particle of the pharmaceutical aerosols 20 can be mass average aerodynamic diameter.

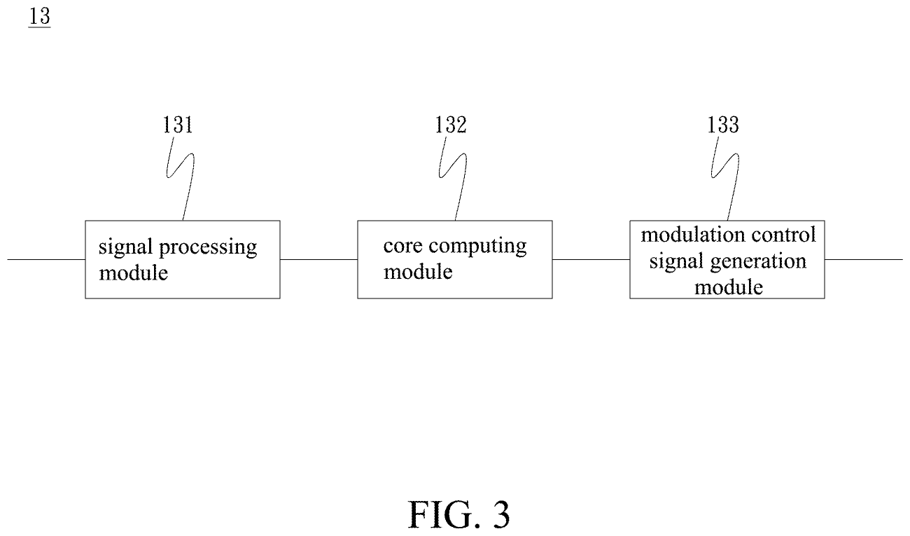

Next, refer to FIG. 2, and FIG. 2 is a schematic diagram showing function blocks of an atomizer according to an embodiment of the present disclosure. In addition to the sensor module 12 and the nozzle 11 shown in FIG. 1, the atomizer 10 further comprises a waveform pattern determination module 13, a data transmission interface DI, a high-frequency oscillation circuit 15, a direct current/alternative current (DC/AC) converter 16, a power amplifier 17, a signal modulation module 14, a piezoelectric device 18, a container CT and a porous screen 19 (p.s., the porous screen 19 is shown in FIG. 3, but not shown in FIG. 2). The waveform pattern determination module 13 is electrically connected to the sensor module 12, the data transmission interface DI is electrically connected to the waveform pattern determination module 13, the signal modulation module 14 is electrically connected to the waveform pattern determination module 13 and the high-frequency oscillation circuit 15, the DC/AC converter 16 is electrically connected to the high-frequency oscillation circuit 15, and the piezoelectric device 18 is electrically connected to the signal modulation module 14 via the power amplifier 17. The porous screen 19 is physically connected to the piezoelectric device 18, and the porous screen 19 and the piezoelectric device 18 are disposed in the container CT.

The sensor module 12 is used for sensing the body state of the patient, wherein the body state includes the breathing state. The sensor module 12 can be, for example, a microphone module, but the present disclosure is not limited thereto. The body state of the patient can be obtained by the sensing of the sound of the patient's breathing through the microphone module. The sensor module 12 may further include other types of sensors to sense at least one of the patient's facial expression, heartbeat rate, heart rhythm, blood pressure, blood glucose and body temperature, an environment temperature and an environment humidity, and correspondingly, the sensor module 12 may further include at least one of a photographing device, a heartbeat meter, a heart rhythm meter, a blood pressure meter, a blood glucose meter, a thermometer, a hygrometer and an ambient temperature sensor. The data transmission interface DI is used to obtain auxiliary information, and the auxiliary information includes at least one of a medicine drug type and a disease type, wherein the data transmission interface DI can be a wired or wireless communication module, and it is signally connected to an external source of the auxiliary information.

The waveform pattern determination module 13 is configured to generate the modulation control signal according to the body state and the auxiliary information. That is, according to the factors which should be considered, the particle size(s) of the pharmaceutical aerosols or the particle size distribution the pharmaceutical aerosols can be determined. In short, the waveform pattern determination module 13 considers various types of data (even big data) to determine how to generate a vibration signal of a specific waveform pattern to the piezoelectric device 18, and the piezoelectric device 18 vibrates according to the vibration signal, thereby generating the corresponding particle size (for example, the particle size varies from 0.1 μm to 10 μm) or the corresponding particle size distribution of the pharmaceutical aerosols, thereby achieving the precision treatment for the patient. The modulation control signal can be one of a square wave, a triangular wave and a sine wave or the combination of the square wave, the triangular wave and the sine wave (i.e., the modulation control signal can be an arbitrary waveform). The modulation control signal is usually a low-frequency signal, such as a low-frequency signal of 0.05 Hz to 10 Hz, but the present disclosure is not limited thereto. In one of the embodiments, the data transmission interface DI can be removed, and the waveform pattern determination module 13 generates a modulation control signal only according to the body state.

The DC/AC converter 16 is used to convert the alternating current input (AC) voltage into a direct current (DC) input voltage, to provide the DC input voltage to a high-frequency oscillation circuit 15 to generate a high-frequency oscillation signal. The frequency of the high-frequency oscillation signal is corresponding to the voltage level of the DC input voltage, i.e., the high-frequency oscillation circuit 15 is a voltage controlled oscillator. For example, the frequency of the high-frequency oscillation signal may be 100 kHz. The DC/AC converter 16 may be various types of bridge rectifier circuits, and the present disclosure is not limited thereto. In addition, in other embodiments, the DC input voltage may be directly input, and the DC/AC converter 16 may be removed.

The signal modulation module 14 is used to modulate the high-frequency oscillation signal by using the modulation control signal to generate the vibration signal to the piezoelectric device 18. In one of the embodiments, the signal modulation module 14 can be an electronic switch, the modulation control signal is used to control the electronic switch to allow or exhibit the high-frequency oscillation signal to pass the electronic switch, so as to generate the vibration signal. In another embodiment, the signal modulation module 14 includes a mixer and a filter module. The mixer is electrically connected to the high-frequency oscillation circuit 15 and the waveform pattern determination module 13, and the filter module is electrically connected to the mixer and is electrically connected to the piezoelectric device 18 through the power amplifier 17. The mixer is used to mix the modulation control signal and the high-frequency oscillation signal to generate a mixed signal. The filter module is used to perform an intermediate frequency (IF) filtering process on the mixed signal to generate a vibration signal. There are many ways for the signal modulation module 14 to perform modulation. Although the above-mentioned modulation method is described with an example of intensity or amplitude modulation, the modulation manner can be one of intensity or amplitude modulation, phase modulation and frequency modulation or combinations thereof, and the present disclosure is not limited thereto.

The power amplifier 17 is used to amplify the vibration signal and output the amplified vibration signal to the piezoelectric device 18. In one embodiment, if the vibration signal is not affected by noise and the power of the vibration signal is sufficient to make the piezoelectric device 18 vibrate, the power amplifier 17 can be removed. The power amplifier 17 may be, for example, a low noise amplifier or other types of amplifiers, and the present disclosure is not limited thereto.

By the way, please refer to FIG. 1 and FIG. 2 at the same time, the atomizer 10 can further comprise a manual switch P. The manual switch P is electrically connected to the waveform pattern determination module 13, and is arranged on the surface of the shell of atomizer 10, and is used to generate the switch control signal according to manipulation of the patient or a manipulator (for example, nurse or physician) on the manual switch P. For example, the manipulation can be press once, long press for a few seconds, press quickly several times, and so on. The waveform pattern determination module 13 can generate the modulation control signal according to the switch control signal. In this way, the atomizer 10 can not only automatically change the particle size(s) of the pharmaceutical aerosols 20 according to the body state of the patient 30, but also manually change the particle size(s) of the pharmaceutical aerosols 20 by operating the manual switch P. Preferably, in the embodiment of the present disclosure, the priority of the switch control signal is greater than that of the body state, that is, when the atomizer 10 receives the switch control signal, it does not consider the body state of the patient 30, but uses the switch control signal to determine the particle size(s) of the pharmaceutical aerosols 20. However, it is noted that the present disclosure is not limited thereto.

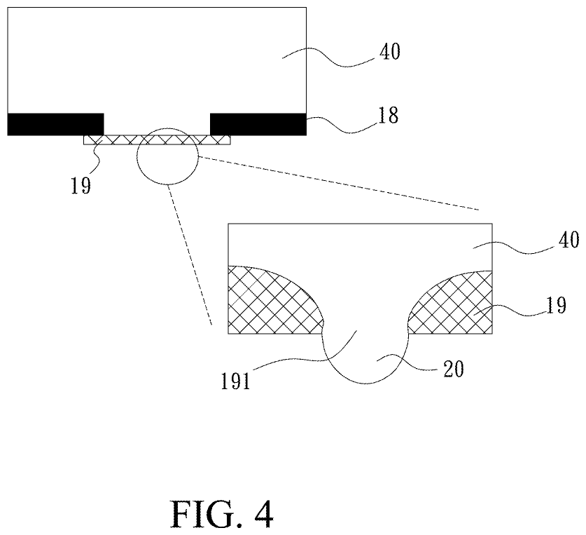

Pease refer to FIG. 2 and FIG. 4 at the same time, the container CT is used to accommodate liquid medicine 40. The piezoelectric device 18 includes, for example, a plurality of piezoelectric sheets, and the porous screen 19 is disposed on any two adjacent piezoelectric sheets. The piezoelectric device 18 is used to generate vibration according to the vibration signal. The porous screen 19 is used to squeeze or press the liquid medicine 40 through the holes 191 of the porous screen 19 according to the vibration of the piezoelectric device 18 to generate a plurality of pharmaceutical aerosols 20, wherein the pharmaceutical aerosols has the particle size(s) corresponding to the modulation control signal. In addition, the nozzle 11 in FIG. 1 is located on the outer surface of the container CT, and is arranged corresponding to the plurality of holes 191 so as to eject or spray the plurality of pharmaceutical aerosols 20.

It is noted that, if it is assumed that the modulation control signal used is a square wave signal, the signal modulation module 14 is implemented by an electronic switch, the high-frequency oscillation signal is a 100 kHz sine wave signal, the square wave signal is a logic high level for 1.5 seconds and a logic low level for 1.5 seconds, the generated pharmaceutical aerosols will have the 3.5 μm MMAD. Further, under the similar condition, but the square wave signal is a logic high level for 1.5 seconds and a logic low level for 3 seconds, and then the generated pharmaceutical aerosols will have the 1.85 μm MMAD. Simply put, by considering various factors to find out the modulation control signal corresponding to the particle size of the pharmaceutical aerosols that can be absorbed by the specific tissue of the patient, the purpose of precision treatment can be achieved.

Refer to FIG. 3, and FIG. 3 is a schematic diagram showing function blocks of a waveform pattern determination module according to an embodiment of the present disclosure. In the embodiment, the waveform pattern determination module 13 includes a signal processing module 131, a core computing module 132 and a modulation control signal generation module 133. The signal processing module 131 is electrically connected to the sensor module 12, the core computing module 132 is electrically connected to the signal processing module 131, and the modulation control signal generation module 133 is electrically connected to the core computing module 132 and the signal modulation module 14.

The signal processing module 131 is used for performing noise filtering processing on the sensing signal of the body state, and in one embodiment, the signal processing module 131 may be an unnecessary element. The core computing module 132 is used to calculate the waveform pattern according to sensing signal on which the noise filtering processing is performed, and the waveform pattern corresponds to the particle size or particle size distribution of the pharmaceutical aerosols. The modulation control signal generation module 133 generates a modulation control signal according to the calculated waveform pattern. In the embodiment of the present invention, the core computing module 132 is a neural network based classifier, so as to calculate the waveform pattern according to the sensing signal processed by noise filtering. In other embodiments, core computing module 132 may be classifiers of other types or core computing modules that perform other specific algorithms.

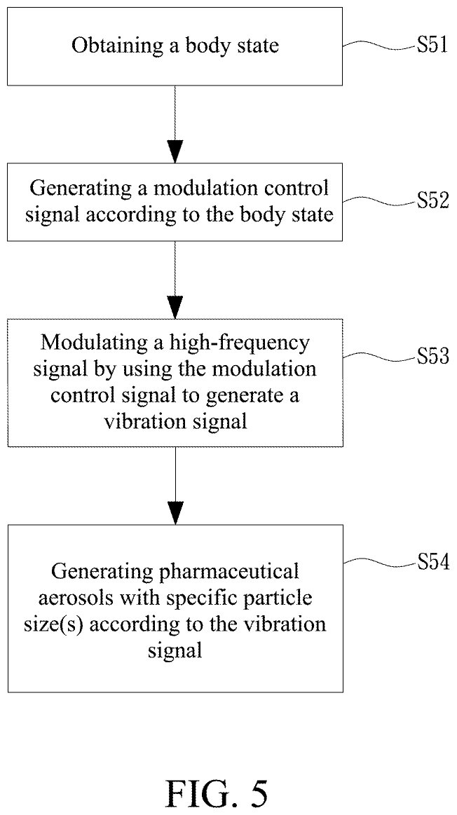

Please refer to FIG. 2, FIG. 4 and FIG. 5, and FIG. 5 is a flow chart of an atomization method according to an embodiment of the present disclosure. The atomization method for adaptively generating the pharmaceutical aerosols with the different particle sizes comprises steps S51 to S54. In step S51, use the sensor module 12 to sense the body state of the patient, wherein the body state includes the breathing state. In step S52, use the waveform pattern determination module 13 to generate the modulation control signal according to the body state. In one embodiment, the atomization method further includes a step of acquiring auxiliary information, and step S52 can be modified to generate a modulation control signal according to the body state and the auxiliary information.

In step S53, use the high-frequency oscillation circuit 15 to provide high-frequency oscillation signal, and use signal modulation module 14 to modulate high-frequency oscillation signal by using modulation control signal to produce the vibration signal. In step S54, the piezoelectric device 18 is used to generate vibration according to the vibration signal, so that the porous screen 19 squeezes and presses the liquid medicine 40 through the holes 191 of the porous screen 19 to generate the plurality of pharmaceutical aerosols 20 with the particle size(s), and the particle size(s) is corresponding to the modulation control signal. Further, the atomization method may comprise other steps which the above atomizer performs, and the present disclosure is not limited thereto.

To sum up, for the body state of different patients, for different diseases, for different medicaments, for factors such as different humidity or temperature, the atomizer and atomization method of the embodiment of the present disclosure can dynamically produce the pharmaceutical aerosols with different particle sizes, to achieve the purpose of precision treatment.

To sum up, the atomizer and the atomization method for adaptively generating the plurality of pharmaceutical aerosols with different particle sizes, both of which are provided by the above embodiments of the present disclosure, can achieve the mentioned technical results. The Applicant believes the atomizer and the atomization method of the present disclosure are not anticipated by prior art, and meet the provision of patentability in the patent act, and allowance of the present disclosure is requested respectfully,

The above-mentioned descriptions represent merely the exemplary embodiment of the present disclosure, without any intention to limit the scope of the present disclosure thereto. Various equivalent changes, alternations or modifications based on the claims of present disclosure are all consequently viewed as being embraced by the scope of the present disclosure.

What is claimed is:

1. An atomizer for adaptively generating a plurality of pharmaceutical aerosols with deferment particle sizes, comprising:
a sensor module, configured to sense a body state of a patient, wherein the body state comprises a breathing state;
a waveform pattern determination module, electrically connected to the sensor module, configured to generate a modulation control signal according to the body state;
a high-frequency oscillation circuit, configured to provide a high-frequency oscillation signal;
a signal modulation module, electrically connected to the waveform pattern determination module and the high-frequency oscillation circuit, configured to module the high-frequency oscillation signal by using the modulation control signal to generate a vibration signal;
a piezoelectric device, electrically connected to the signal modulation module, configured to vibrate according to the vibration signal; and
a porous screen, physically connected to the piezoelectric device, configured to presses liquid medicine through a plurality of holes of the porous screen to generate the plurality of pharmaceutical aerosols according to vibration of the piezoelectric device, wherein the pharmaceutical aerosols has the particle size(s) corresponding to the modulation control signal.

2. The atomizer of claim 1, further comprising:
a data transmission interface, electrically connected to the waveform pattern determination module, configured to obtain auxiliary information;
wherein the signal modulation module generates the modulation control signal according to the body state and the auxiliary information, the auxiliary information comprises at least one of a medicine drug type and a disease type, and the body state further comprises at least of a facial expression, a heartbeat rate, a heart rhythm, a blood pressure, a glucose and body temperature of the patient.

3. The atomizer of claim 1, further comprising:
a manual switch, electrically connected to the waveform pattern determination module, configured to generate a switch control signal according to manipulation of the patient or a manipulator, wherein the waveform pattern determination module generates the modulation control signal according to the switch control signal.

4. The atomizer of claim 1, further comprising:
a direct current/alternative current (DC/AC) converter, electrically connected to the high-frequency oscillation circuit, configured to convert an AC input voltage to a DC input voltage, so as to provide the DC input voltage to the high-frequency oscillation circuit, wherein a frequency of the high-frequency oscillation signal generated by the high-frequency oscillation circuit is corresponding to the DC input voltage; and
a power amplifier, electrically connected between the piezoelectric device and the signal modulation module, configured to amplify the vibration signal and output the amplified vibration signal to the piezoelectric device.

5. The atomizer of claim 1, wherein the waveform pattern determination module comprises:
a signal processing module, electrically connected to the sensor module, configured to perform noise filtering process on a sensing signal of the body state;
a core computing module, electrically connected to the signal processing module, configured to calculate a waveform pattern according to the sensing signal on which the noise filtering process is performed; and
a modulation control signal generation module, electrically connected to the core computing module and the signal modulation module, configured to generate the modulation control signal according to the calculated waveform pattern.

6. The atomizer of claim 5, wherein the core computing module comprises a neural network based classifier, and the neural network based classifier is configured to calculate a waveform pattern according to the sensing signal on which the noise filtering process is performed.

7. The atomizer of claim 1, wherein the sensor module comprises a microphone module.

8. The atomizer of claim 1, further comprising:
a container, configured to accommodate the liquid medicine; and
a nozzle, disposed on an outer surface of the container and arranged corresponding to the holes, configured to spray the plurality of the pharmaceutical aerosols.

9. The atomizer of claim 1, wherein the signal modulation module comprises:
a mixer, electrically connected to the high-frequency oscillation circuit and the waveform pattern determination module, configured to mix the modulation control signal and the high-frequency oscillation signal to generate a mixed signal; and
a filter module filter module, electrically connected to the mixer and the piezoelectric device, configured to perform an intermediate frequency filtering process on the mixed signal to generate the vibration signal.

10. The atomizer of claim 1, wherein the modulation control signal is one or combination of a square wave, a triangle wave and a sinuous wave.

11. An atomization method for adaptively generating a plurality of pharmaceutical aerosols with deferment particle sizes, comprising:
using a sensor module to sense a body state of a patient, wherein the body state comprises a breathing state;
using a waveform pattern determination module to generate a modulation control signal according to the body state;
using a high-frequency oscillation circuit to provide a high-frequency oscillation signal;
using a signal modulation module to module the high-frequency oscillation signal by using the modulation control signal to generate a vibration signal; and
using a piezoelectric device to vibrate according to the vibration signal so as to make a porous screen presses liquid medicine through a plurality of holes of the porous screen to generate the plurality of pharmaceutical aerosols according to vibration of the piezoelectric device, wherein the pharmaceutical aerosols has the particle size(s) corresponding to the modulation control signal.

12. The atomization method of claim 11, further comprising:
using a data transmission interface to obtain auxiliary information;
wherein the signal modulation module generates the modulation control signal according to the body state and the auxiliary information, the auxiliary information comprises at least one of a medicine drug type and a disease type, and the body state further comprises at least of a facial expression, a heartbeat rate, a heart rhythm, a blood pressure, a glucose and body temperature of the patient.

13. The atomization method of claim 11, further comprising:
using a manual switch to generate a switch control signal according to manipulation of the patient or a manipulator, wherein the waveform pattern determination module generates the modulation control signal according to the switch control signal.

14. The atomization method of claim 11, further comprising:
using a direct current/alternative current (DC/AC) converter to convert an AC input voltage to a DC input voltage, so as to provide the DC input voltage to the high-frequency oscillation circuit, wherein a frequency of the high-frequency oscillation signal generated by the high-frequency oscillation circuit is corresponding to the DC input voltage; and
using a power amplifier to amplify the vibration signal and output the amplified vibration signal to the piezoelectric device.

15. The atomization method of claim 11, wherein the step of using the waveform pattern determination module to generate the modulation control signal according to the body state comprises:
using a signal processing module to perform noise filtering process on a sensing signal of the body state;
using a core computing module to calculate a waveform pattern according to the sensing signal on which the noise filtering process is performed; and
using a modulation control signal generation module to generate the modulation control signal according to the calculated waveform pattern.

16. The atomization method of claim 15, wherein the core computing module comprises a neural network based classifier, and the neural network based classifier is configured to calculate a waveform pattern according to the sensing signal on which the noise filtering process is performed.

17. The atomization method of claim 11, wherein the sensor module comprises a microphone module.

18. The atomization method of claim 11, further comprising:
using a container to accommodate the liquid medicine; and
using a nozzle to spray the plurality of the pharmaceutical aerosols.

19. The atomization method of claim 11, wherein the step of using the signal modulation module to module the high-frequency oscillation signal by using the modulation control signal to generate the vibration signal comprises:
using a mixer to mix the modulation control signal and the high-frequency oscillation signal to generate a mixed signal; and
using a filter module filter module to perform an intermediate frequency filtering process on the mixed signal to generate the vibration signal.

20. The atomization method of claim 11, wherein the modulation control signal is one or combination of a square wave, a triangle wave and a sinuous wave.
